(19) United States
(12) Patent Application Publication
(10) Pub. No.: US 2018/0235573 A1
(43) Pub. Date: Aug. 23, 2018

(54) SYSTEMS AND METHODS FOR INTERVENTION GUIDANCE USING A COMBINATION OF ULTRASOUND AND X-RAY IMAGING

(71) Applicant: General Electric Company, Schenectady, NY (US)

(72) Inventors: Stian Langeland, Vollen (NO); Eigil Samset, Oppegard (NO); Olivier Gerard, Oslo (NO)

(21) Appl. No.: 15/438,407

(22) Filed: Feb. 21, 2017

(51) Int. Cl. A61B 8/00; A61B 6/00; A61B 5/055; A61B 6/03; A61B 5/00; A61B 8/08

(52) U.S. Cl. CPC A61B 8/4416; A61B 6/4417; A61B 6/463; A61B 5/055; A61B 6/032; A61B 2090/365; A61B 5/0035; A61B 6/5247; A61B 8/5261; A61B 8/463; A61B 8/0841; A61B 5/7425

(57) ABSTRACT

Methods and systems are provided for multi-modality imaging. In one embodiment, a method comprises: during an ultrasound scan of a patient, co-aligning an ultrasound image received during the ultrasound scan with a three-dimensional (3D) image of the patient acquired with an imaging modality prior to the ultrasound scan; calculating an angle for an x-ray source based on position information in the 3D image to align the x-ray source with the ultrasound image; and adjusting a position of the x-ray source based on the calculated angle. In this way, the same internal views of a patient may be obtained with multiple modalities during an intervention with minimal user input.

### FIELD

Embodiments of the subject matter disclosed herein relate to multi-modality imaging, and more particularly, to interventional cardiology.

### BACKGROUND

Presently available medical imaging technologies such as ultrasound imaging, magnetic resonance imaging (MRI), computed tomography (CT) imaging, and x-ray fluoroscopic imaging are known to be helpful not only for non-invasive diagnostic purposes, but also for providing assistance during surgery. For example, during cardiac interventions, ultrasound imaging is often utilized for guidance and monitoring of the procedure. X-ray angiography may also be used in conjunction with ultrasound during cardiac interventions to provide additional guidance. Ultrasound images include more anatomical information of cardiac structures than x-ray images which do not effectively depict soft structures, while x-ray images more effectively depict catheters and other surgical instruments than ultrasound images.

### BRIEF DESCRIPTION

In one embodiment, a method comprises: during an ultrasound scan of a patient, co-aligning an ultrasound image received during the ultrasound scan with a three-dimensional image of the patient acquired with an imaging modality prior to the ultrasound scan; calculating an angle for an x-ray source based on position information in the three-dimensional image to align the x-ray source with the ultrasound image; and adjusting a position of the x-ray source based on the calculated angle. In this way, the same or related anatomical views of a patient may be obtained with multiple modalities during an intervention with minimal user input.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 illustrates a multi-modality imaging system including an ultrasound system and an x-ray fluoroscopic system formed in accordance with an embodiment;

FIG. 2 shows a computed tomography (CT) imaging system in accordance with an embodiment; and

FIG. 3 shows a high-level flow chart illustrating an example method for positioning an x-ray device during an ultrasound scan in accordance with an embodiment.

### DETAILED DESCRIPTION

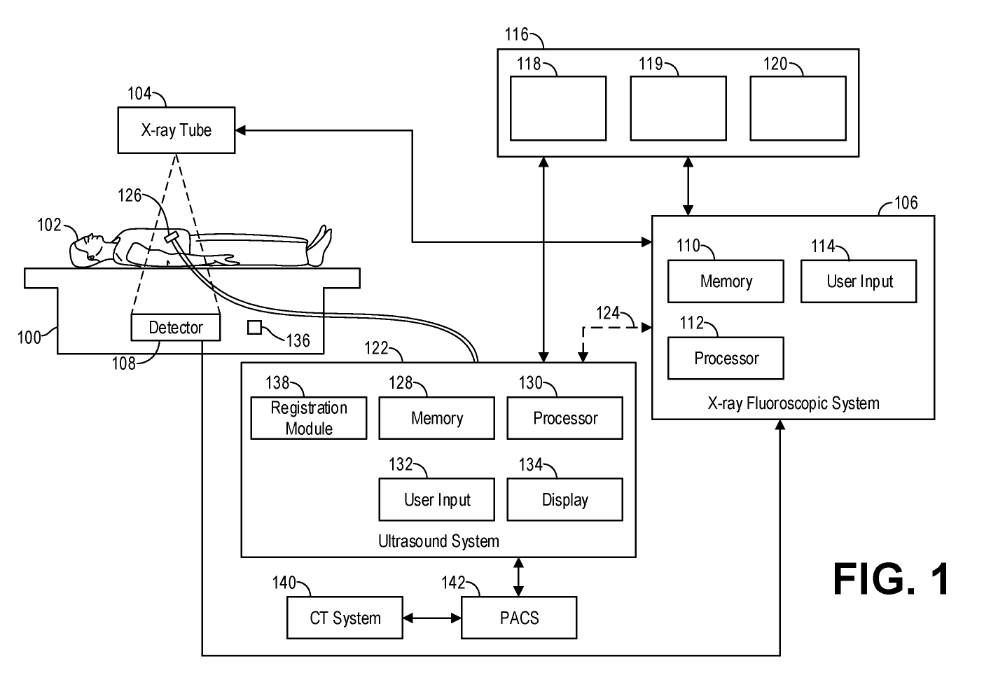

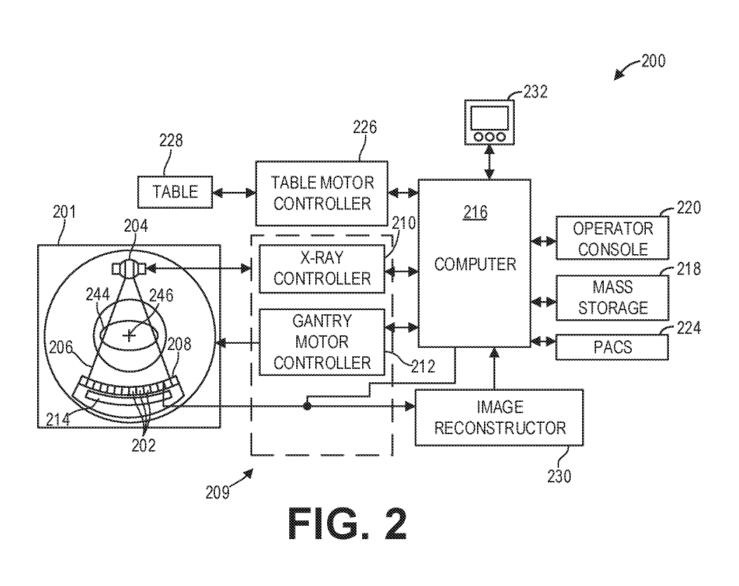

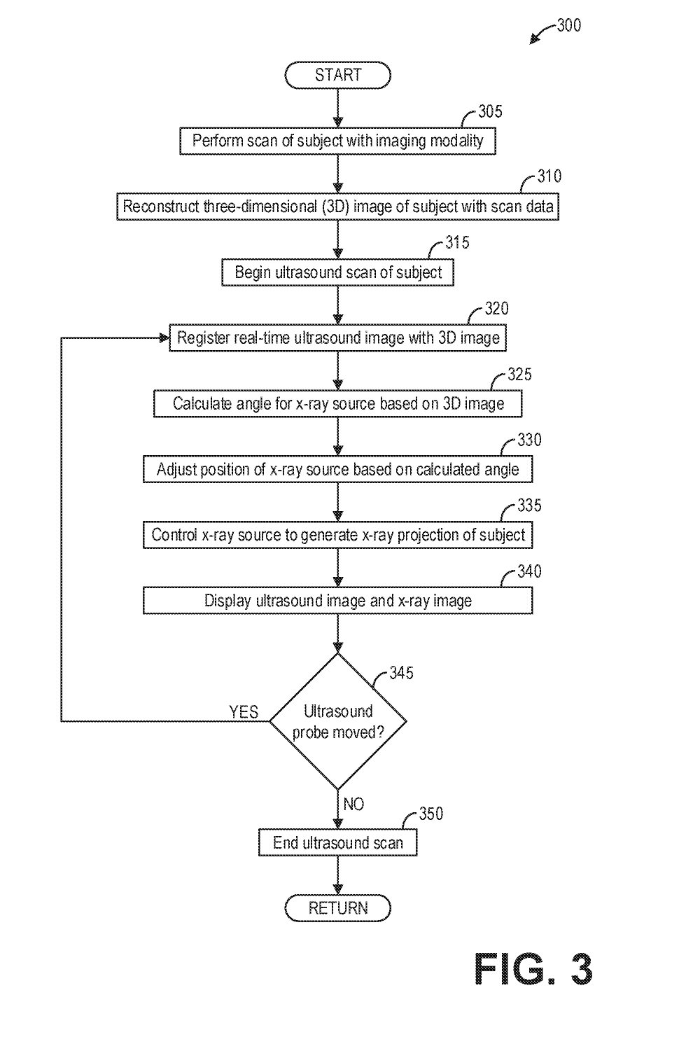

The following description relates to various embodiments of multi-modality imaging. In particular, systems and methods are provided for intervention guidance using both ultrasound and x-ray imaging for interventional cardiology. A multi-modality imaging system for interventional procedures, such as the system depicted in FIG. 1, may include multiple imaging modalities, including but not limited to computed tomography (CT), ultrasound, and x-ray fluoroscopy. Pre-operative diagnostic images may be acquired with a CT imaging system, such as the CT imaging system depicted in FIG. 2. A method for acquiring the same view with an x-ray fluoroscopy system as an ultrasound system, such as the method depicted in FIG. 3, may include registering a pre-operative CT image with an ultrasound image. Projection angles for the x-ray fluoroscopy system may be obtained based on the ultrasound slices, given that the ultrasound image is registered with the pre-operative CT image.

Though a CT system is described by way of example for acquiring pre-operative diagnostic images, it should be understood that the present techniques may also be useful when applied to images acquired using other three-dimensional imaging modalities, such as MRI, PET, SPECT, and so forth. The present discussion of a CT imaging modality for acquiring pre-operative diagnostic images is provided merely as an example of one suitable imaging modality.

FIG. 1 illustrates a multi-modality imaging system 10 in accordance with an embodiment of the present invention. Multi-modality imaging system 10 may include an x-ray fluoroscopic system 106, an ultrasound system 122, and a computed tomography (CT) system 140. An example CT system is described further herein with regard to FIG. 2.

A table 100 or bed is provided for supporting a subject 102. An x-ray tube 104 or other generator is connected to an x-ray fluoroscopic system 106. As shown, the x-ray tube 104 is positioned above the subject 102, but it should be understood that the x-ray tube 104 may be moved to other positions with respect to the subject 102. A detector 108 is positioned opposite the x-ray tube 104 with the subject 102 there-between. The detector 108 may be any known detector capable of detecting x-ray radiation.

The x-ray fluoroscopic system 106 has at least a memory 110, a processor 112, and at least one user input 114, such as a keyboard, trackball, pointer, touch panel, and the like. To acquire an x-ray image, the x-ray fluoroscopic system 106 causes the x-ray tube 104 to generate x-rays and the detector 108 detects an image. Fluoroscopy may be accomplished by activating the x-ray tube 104 continuously or at predetermined intervals while the detector 108 detects corresponding images. Detected image(s) may be displayed on a display 116 that may be configured to display a single image or more than one image at the same time.

In some examples, the ultrasound system 122 communicates with the x-ray fluoroscopic system 106 via an optional connection 124. The connection 124 may be a wired or wireless connection. The ultrasound system 122 may transmit or convey ultrasound imaging data to the x-ray fluoroscopic system 106. The communication between the systems 106 and 122 may be one-way or two-way, allowing image data, commands, and information to be transmitted between the two systems 106 and 122. The ultrasound system 122 may be a stand-alone system that may be moved from room to room, such as a cart-based system, hand-carried system, or other portable system.

An operator (not shown) may position an ultrasound probe 126 on the subject 102 to image an area of interest within the subject 102. The ultrasound system 122 has at least a memory 128, a processor 130, and a user input 132. Optionally, if the ultrasound system 122 is a stand-alone system, a display 134 may be provided. By way of example, images acquired using the x-ray fluoroscopic system 106 may be displayed as a first image 118 and images acquired using the ultrasound system 122 may be displayed as a second image 120 on the display 116, forming a dual display configuration. In another embodiment, two side-by-side monitors (not shown) may be used. The images acquired by both the x-ray fluoroscopic system 106 and the ultrasound system 122 may be acquired in known manners.

In one embodiment, the ultrasound system 122 may be a 3D-capable miniaturized ultrasound system that is connected to the x-ray fluoroscopic system 106 via the connection 124. As used herein, “miniaturized” means that the ultrasound system 122 is configured to be carried in a person's hand, pocket, briefcase-sized case, or backpack. For example, the ultrasound system 122 may be a hand-carried device having a size of a typical laptop computer, for instance, having dimensions of approximately 2.5 inches in depth, approximately 14 inches in width, and approximately 12 inches in height. The ultrasound system 122 may weigh approximately ten pounds, and thus is easily portable by the operator. An integrated display, such as the display 134, may be configured to display an ultrasound image as well as an x-ray image acquired by the x-ray fluoroscopic system 106.

As another example, the ultrasound system 122 may be a 3D capable pocket-sized ultrasound system. By way of example, the pocket-sized ultrasound system may be approximately 2 inches wide, approximately 4 inches in length, and approximately 0.5 inches in depth, and weigh less than 3 ounces. The pocket-sized ultrasound system may include a display (e.g., the display 134), a user interface (e.g., user input 132), and an input/output (I/O) port for connection to the probe 126. It should be noted that the various embodiments may be implemented in connection with a miniaturized or pocket-sized ultrasound system having different dimensions, weights, and power consumption.

In another embodiment, the ultrasound system 122 may be a console-based ultrasound imaging system provided on a movable base. The console-based ultrasound imaging system may also be referred to as a cart-based system. An integrated display (e.g., the display 134) may be used to the display the ultrasound image alone or simultaneously with the x-ray image as discussed herein.

In yet another embodiment, the x-ray fluoroscopic system 106 and the ultrasound system 122 may be integrated together and may share at least some processing, user input, and memory functions. For example, a probe port 136 may be provided on the table 100 or other apparatus near the subject 102. The probe 126 may thus be connected to the probe port 136.

In some examples, a CT image 119 of the patient 102 may be acquired with the CT system 140. The CT system 140 may include or may be coupled to a picture archiving and communications system (PACS) 142. As depicted, the ultrasound system 122 may also be coupled to the PACS 142. As described further herein with regard to FIG. 3, the ultrasound system 122 may include a registration module 138 configured to register the ultrasound image 118 and the CT image 119 retrieved from the PACS 142 with respect to each other. As described further herein with regard to FIG. 3, one or more projection angles may be calculated based on the co-aligned ultrasound image 118 and the CT image 119, and these projection angles may be used to position the x-ray source 126 such that a subsequently acquired x-ray projection image 120 provides a same view as the ultrasound image 118 or a view related to the view of the ultrasound image 118.

FIG. 2 illustrates an exemplary computed tomography (CT) imaging system 200 configured to allow fast and iterative image reconstruction. Particularly, the CT system 200 is configured to image a subject such as a patient, an inanimate object, one or more manufactured parts, and/or foreign objects such as dental implants, stents, and/or contrast agents present within the body. The CT system 200 may be implemented in the multi-modality imaging system 10 as CT system 140.

In one embodiment, the CT system 200 includes a gantry 201, which in turn, may further include at least one x-ray radiation source 204 configured to project a beam of x-ray radiation 206 for use in imaging the patient. Specifically, the radiation source 204 is configured to project the x-rays 206 towards a detector array 208 positioned on the opposite side of the gantry 201. Although FIG. 2 depicts only a single radiation source 204, in certain embodiments, multiple radiation sources may be employed to project a plurality of x-rays 206 for acquiring projection data corresponding to the patient at different energy levels.

In one embodiment, the system 200 includes the detector array 208. The detector array 208 further includes a plurality of detector elements 202 that together sense the x-ray beams 206 that pass through a subject 244 such as a patient to acquire corresponding projection data. Accordingly, in one embodiment, the detector array 208 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the system 200 is configured to traverse different angular positions around the subject 244 for acquiring desired projection data. Accordingly, the gantry 201 and the components mounted thereon may be configured to rotate about a center of rotation 246 for acquiring the projection data, for example, at different energy levels. Alternatively, in embodiments where a projection angle relative to the subject 244 varies as a function of time, the mounted components may be configured to move along a general curve rather than along a segment of a circle.

In one embodiment, the system 200 includes a control mechanism 209 to control movement of the components such as rotation of the gantry 201 and the operation of the x-ray radiation source 204. In certain embodiments, the control mechanism 209 further includes an x-ray controller 210 configured to provide power and timing signals to the radiation source 204. Additionally, the control mechanism 209 includes a gantry motor controller 212 configured to control a rotational speed and/or position of the gantry 201 based on imaging requirements.

In certain embodiments, the control mechanism 209 further includes a data acquisition system (DAS) 214 configured to sample analog data received from the detector elements 202 and convert the analog data to digital signals for subsequent processing. The data sampled and digitized by the DAS 214 is transmitted to a computing device 216. In one example, the computing device 216 stores the data in a storage device 218. The storage device 218, for example, may include a hard disk drive, a floppy disk drive, a compact disk-read/write (CD-R/W) drive, a Digital Versatile Disc (DVD) drive, a flash drive, and/or a solid-state storage device.

Additionally, the computing device 216 provides commands and parameters to one or more of the DAS 214, the x-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) and/or a touchscreen to allow the operator to specify the commands and/or scanning parameters.

Although FIG. 2 illustrates only one operator console 220, more than one operator console may be coupled to the system 200, for example, for inputting or outputting system parameters, requesting examinations, and/or viewing images. Further, in certain embodiments, the system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks.

In one embodiment, for example, the system 200 either includes, or is coupled to a picture archiving and communications system (PACS) 224, which may comprise the PACS 142 described hereinabove with regard to FIG. 1. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-define commands and parameters to operate a table motor controller 226, which in turn, may control a motorized table 228. Particularly, the table motor controller 226 moves the table 228 for appropriately positioning the subject 244 in the gantry 201 for acquiring projection data corresponding to the target volume of the subject 244.

As previously noted, the DAS 214 samples and digitizes the projection data acquired by the detector elements 202. Subsequently, an image reconstructor 230 uses the sampled and digitized x-ray data to perform high-speed reconstruction. In certain embodiments, the image reconstructor 230 is configured to reconstruct images of a target volume of the patient using an iterative or analytic image reconstruction method. For example, the image reconstructor 230 may use an analytic image reconstruction approach such as filtered backprojection (FBP) to reconstruct images of a target volume of the patient. As another example, the image reconstructor 230 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the patient.

Although FIG. 2 illustrates the image reconstructor 230 as a separate entity, in certain embodiments, the image reconstructor 230 may form part of the computing device 216. Alternatively, the image reconstructor 230 may be absent from the system 200 and instead the computing device 216 may perform one or more functions of the image reconstructor 230. Moreover, the image reconstructor 230 may be located locally or remotely, and may be operatively connected to the system 200 using a wired or wireless network. Particularly, one exemplary embodiment may use computing resources in a “cloud” network cluster for the image reconstructor 230.

In one embodiment, the image reconstructor 230 stores the reconstructed images in the storage device 218. Alternatively, the image reconstructor 230 transmits the reconstructed images to the computing device 216 for generating useful patient information for diagnosis and evaluation. In certain embodiments, the computing device 216 transmits the reconstructed images and/or the patient information to a display 232 communicatively coupled to the computing device 216 and/or the image reconstructor 230.

The various methods and processes described further herein may be stored as executable instructions in non-transitory memory on a computing device in system 200. In one embodiment, image reconstructor 230 may include such instructions in non-transitory memory, and may apply the methods described herein to reconstruct an image from scan data. In another embodiment, computing device 216 may include the instructions in non-transitory memory, and may apply the methods described herein, at least in part, to a reconstructed image after receiving the reconstructed image from image reconstructor 230. In yet another embodiment, the methods and processes described herein may be distributed across image reconstructor 230 and computing device 216.

In one embodiment, the display 232 allows the operator to evaluate the imaged anatomy. The display 232 may also allow the operator to select a volume of interest (VOI) and/or request patient information, for example, via graphical user interface (GUI) for a subsequent scan or processing.

FIG. 3 shows a high-level flow chart illustrating an example method 300 for interventional guidance using a combination of ultrasound and x-ray imaging. In particular, method 300 relates to adjusting the position of an x-ray source on a C-arm imaging device to align the x-ray projections with live ultrasound slices. Method 300 may be carried out using the systems and components described hereinabove with regard to FIGS. 1-2, though it should be understood that the method may be implemented with other systems and components without departing from the scope of the present disclosure.

Method 300 begins at 305. At 305, method 300 performs a scan of a subject with an imaging modality, for example using a CT imaging system such as the CT system 140 or the CT imaging system 200 described hereinabove with regard to FIG. 2. In some examples, method 300 performs a scan of the subject with an imaging modality such as a magnetic resonance imaging (MRI) system, or any suitable imaging modality configured to generate a three-dimensional image of the patient's anatomy. At 310, method 300 reconstructs a three-dimensional (3D) image of the subject using data acquired during the scan. For examples wherein a CT imaging system is used to perform the scan at 305, method 300 may reconstruct a CT image of the subject using any suitable image reconstruction algorithm, such as filtered backprojection or an iterative reconstruction algorithm. Similarly, for examples wherein an MRI imaging system is used to perform the scan at 305, method 300 may reconstruct an MRI image of the subject.

Continuing at 315, method 300 begins an ultrasound scan of the subject, for example using the ultrasound system 122. It should be appreciated that the subject may be positioned similarly during the scan at 305 and the ultrasound scan, for example, the subject or patient may lay on their back on an imaging table.

At 320, method 300 registers the real-time ultrasound image with the 3D image. In some examples, method 300 may automatically register the real-time ultrasound image with the 3D image. In other examples, the live ultrasound image may be manually registered with the 3D image. For example, one or more anatomical landmarks may be manually identified by a user in both the ultrasound image and the 3D image. Method 300 may then register the images based on the identified landmarks.

At 325, method 300 calculates an angle for the x-ray source based on the 3D image. The 3D image contains information regarding how the 3D image is acquired based on position of the patient. Since the 3D image and the ultrasound image are registered, the accurate position information of the 3D image may be used to calculate a desired position for the x-ray source such that the x-ray beam emitted by the x-ray source is in the same direction as the ultrasound probe.

At 330, method 300 adjusts the position of the x-ray source based on the calculated angle. In some examples, the method may display the calculated angle or position via a display device such as display 134, and the user may input the calculated angle into the user input (e.g., user input 114) of the C-arm imaging system or x-ray system to adjust the position of the x-ray source. In other examples, the method may automatically adjust the position of the x-ray source based on the calculated angle (e.g., without user input or intervention). As an illustrative example, the ultrasound system 122 may provide a command, via connection 124, to the x-ray fluoroscopic system 106 to adjust the position of the x-ray tube 126.

At 335, method 300 controls the x-ray source to generate an x-ray projection of the subject. The x-ray source generates an x-ray beam that passes through the subject, and the detector receives the x-rays attenuated by the subject. The x-ray projection thus generated is parallel to the ultrasound slice of the real-time ultrasound image. In this way, the user performing the intervention may utilize both the real-time ultrasound image and the static x-ray image for guidance, without the need to manually reposition the x-ray source. At 340, method 300 displays the ultrasound image and the x-ray image, for example via a display device.

At 345, method 300 determines if the ultrasound probe is moved. In some examples, the method may automatically determine if the ultrasound probe is moved. In other examples, the user may manually indicate, for example via user input 132 of the ultrasound system 122, that the probe is moved so that re-registration may be performed.

If the ultrasound probe is moved (“YES”), method 300 returns to 320. The ultrasound image acquired from the new position of the ultrasound probe and the 3D image may be registered, and the method continues as described above. However, if the ultrasound probe is not moved (“NO”), method 300 proceeds to 350, wherein method 300 ends the ultrasound scan. Method 300 then returns.

A technical effect of the disclosure is the calculation of a desired x-ray view based on live ultrasound images co-registered with pre-operative CT images. Another technical effect of the disclosure is the display of x-ray projection angles that best depicts certain anatomical structures as seen by the ultrasound imaging device. Another technical effect of the disclosure is the acquisition of an x-ray projection at a same angle as an ultrasound imaging device. Yet another technical effect of the disclosure is the automatic positioning of an x-ray source based on an angle obtained from a live ultrasound image.

In one embodiment, a method comprises: during an ultrasound scan of a patient, co-aligning an ultrasound image received during the ultrasound scan with a three-dimensional (3D) image of the patient acquired with an imaging modality prior to the ultrasound scan; calculating an angle for an x-ray source based on position information in the 3D image to align the x-ray source with the ultrasound image; and adjusting a position of the x-ray source based on the calculated angle.

In a first example of the method, the ultrasound image is manually co-aligned with the 3D image responsive to a user indicating one or more landmarks in both the ultrasound image and the 3D image. In a second example of the method optionally including the first example, the ultrasound image is automatically co-aligned with the 3D image. In a third example of the method optionally including one or more of the first and second examples, the x-ray source is mounted on a C-arm opposite a detector, and adjusting the position of the x-ray source comprises adjusting an orientation of the C-arm. In a fourth example of the method optionally including one or more of the first through third examples, the method further comprises controlling the x-ray source to generate an x-ray projection of the patient, wherein the x-ray projection is parallel to a plane of the ultrasound image. In a fifth example of the method optionally including one or more of the first through fourth examples, the method further comprises displaying the x-ray projection and the ultrasound image via a display device. In a sixth example of the method optionally including one or more of the first through fifth examples, the 3D image is acquired with the imaging modality while the patient is in a same orientation as during the ultrasound scan, and the imaging modality comprises one of a computed tomography (CT) system or a magnetic resonance imaging (MM) system. In a seventh example of the method optionally including one or more of the first through sixth examples, the method further comprises, responsive to an updated position of an ultrasound probe during the ultrasound scan, co-aligning the 3D image with an ultrasound image generated by the ultrasound probe in the updated position. In an eighth example of the method optionally including one or more of the first through seventh examples, the method further comprises displaying the calculated angle via a display device, and wherein adjusting the position of the x-ray source comprises receiving a user input regarding the calculated angle and controlling an arm mounting the x-ray source to move to the adjusted position. In a ninth example of the method optionally including one or more of the first through eighth examples, the x-ray source is automatically adjusted to the position indicated by the calculated angle.

In another embodiment, a method comprises: retrieving a three-dimensional computed tomography (CT) image of a patient; acquiring, with an ultrasound probe, a three-dimensional ultrasound image of the patient; registering the three-dimensional CT image with the three-dimensional ultrasound image; adjusting, based on position data in the three-dimensional CT image, an angle of an x-ray imaging arm containing an x-ray source and a detector to align the x-ray source with the ultrasound probe; and acquiring, with the x-ray imaging arm, a two-dimensional x-ray projection of the patient.

In a first example of the method, the two-dimensional x-ray projection is parallel to a plane of the ultrasound probe. In a second example of the method optionally including the first example, the CT image is acquired via a CT imaging system while the patient is oriented in a same orientation as during the acquisition of the ultrasound image.

In yet another embodiment, a system comprises: an x-ray imaging arm containing an x-ray source and detector; an ultrasound probe; and a processor communicatively coupled to the ultrasound probe, the processor configured with instructions in non-transitory memory that when executed cause the processor to: during an ultrasound scan with the ultrasound probe of a subject, co-align an ultrasound image received during the ultrasound scan with a three-dimensional (3D) image of the subject acquired with an imaging modality prior to the ultrasound scan; calculate an angle for the x-ray source based on position information in the 3D image to align the x-ray source with the ultrasound image; and adjust a position of the x-ray source based on the calculated angle.

In a first example of the system, the ultrasound image is manually co-aligned with the 3D image responsive to a user indicating, via a user interface communicatively coupled to the processor, one or more landmarks in both the ultrasound image and the 3D image. In a second example of the system optionally including the first example, the processor is further configured with instructions in the non-transitory memory that when executed cause the processor to control the x-ray source to generate an x-ray projection of the subject, wherein the x-ray projection is parallel to a plane of the ultrasound image. In a third example of the system optionally including one or more of the first and second examples, the 3D image is acquired with the imaging modality while the subject is in a same orientation as during the ultrasound scan, and the imaging modality comprises one of a CT imaging system or an MRI system. In a fourth example of the system optionally including one or more of the first through third examples, the system further comprises a display device communicatively coupled to the processor, wherein the processor is further configured with instructions in the non-transitory memory that when executed cause the processor to display the x-ray projection and the ultrasound image via a display device. In a fifth example of the system optionally including one or more of the first through fourth examples, the processor is further configured to, responsive to an updated position of the ultrasound probe during the ultrasound scan, co-align the 3D image with an ultrasound image generated by the ultrasound probe in the updated position. In a sixth example of the system optionally including one or more of the first through fifth examples, the processor is communicatively coupled to the x-ray source and the detector, and the x-ray source is automatically adjusted to the position indicated by the calculated angle.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
during an ultrasound scan of a patient, co-aligning an ultrasound image received during the ultrasound scan with a three-dimensional (3D) image of the patient acquired with an imaging modality prior to the ultrasound scan;
calculating an angle for an x-ray source based on position information in the 3D image to align the x-ray source in relation to the ultrasound image; and
adjusting a position of the x-ray source based on the calculated angle.

2. The method of claim 1, wherein the ultrasound image is manually co-aligned with the 3D image responsive to a user indicating one or more landmarks in both the ultrasound image and the 3D image.

3. The method of claim 1, wherein the ultrasound image is automatically co-aligned with the 3D image.

4. The method of claim 1, wherein the x-ray source is mounted on a C-arm opposite a detector, and wherein adjusting the position of the x-ray source comprises adjusting an orientation of the C-arm.

5. The method of claim 1, further comprising controlling the x-ray source to generate an x-ray projection of the patient, wherein the x-ray projection is parallel to a plane of the ultrasound image.

6. The method of claim 1, further comprising displaying the x-ray projection and the ultrasound image via a display device.

7. The method of claim 1, wherein the 3D image is acquired with the imaging modality while the patient is in a same orientation as during the ultrasound scan, the imaging modality comprising one of a computed tomography (CT) imaging system or a magnetic resonance imaging (MM) system.

8. The method of claim 1, further comprising, responsive to an updated position of an ultrasound probe during the ultrasound scan, co-aligning the 3D image with an ultrasound image generated by the ultrasound probe in the updated position.

9. The method of claim 1, further comprising displaying the calculated angle via a display device, and wherein adjusting the position of the x-ray source comprises receiving a user input regarding the calculated angle and controlling an arm mounting the x-ray source to move to the adjusted position.

10. The method of claim 1, wherein the x-ray source is automatically adjusted to the position indicated by the calculated angle.

11. A method, comprising:
retrieving a three-dimensional computed tomography (CT) image of a patient;
acquiring, with an ultrasound probe, a three-dimensional ultrasound image of the patient;
registering the three-dimensional CT image with the three-dimensional ultrasound image;
adjusting, based on position data in the three-dimensional CT image, an angle of an x-ray imaging arm containing an x-ray source and a detector to align the x-ray source with the ultrasound probe; and
acquiring, with the x-ray imaging arm, a two-dimensional x-ray projection of the patient.

12. The method of claim 11, wherein the two-dimensional x-ray projection is parallel to a plane of the ultrasound probe.

13. The method of claim 11, wherein the CT image is acquired via a CT imaging system while the patient is oriented in a same orientation as during the acquisition of the ultrasound image.

14. A system, comprising:
an x-ray imaging arm containing an x-ray source and detector;
an ultrasound probe; and
a processor communicatively coupled to the ultrasound probe, the processor configured with instructions in non-transitory memory that when executed cause the processor to:
during an ultrasound scan with the ultrasound probe of a subject, co-align an ultrasound image received during the ultrasound scan with a three-dimensional (3D) image of the subject acquired with an imaging modality prior to the ultrasound scan;
calculate an angle for the x-ray source based on position information in the 3D image to align the x-ray source with the ultrasound image; and adjust a position of the x-ray source based on the calculated angle.

15. The system of claim 14, wherein the ultrasound image is manually co-aligned with the 3D image responsive to a user indicating, via a user interface communicatively coupled to the processor, one or more landmarks in both the ultrasound image and the 3D image.

16. The system of claim 14, wherein the processor is further configured with instructions in the non-transitory memory that when executed cause the processor to control the x-ray source to generate an x-ray projection of the subject, wherein the x-ray projection is parallel to a plane of the ultrasound image.

17. The system of claim 14, wherein the 3D image is acquired with the imaging modality while the subject is in a same orientation as during the ultrasound scan, wherein the imaging modality comprises one of a computed tomography (CT) imaging system or a magnetic resonance imaging (MM) system.

18. The system of claim 14, further comprising a display device communicatively coupled to the processor, wherein the processor is further configured with instructions in the non-transitory memory that when executed cause the processor to display the x-ray projection and the ultrasound image via a display device.

19. The system of claim 14, wherein the processor is further configured to, responsive to an updated position of the ultrasound probe during the ultrasound scan, co-align the 3D image with an ultrasound image generated by the ultrasound probe in the updated position.

20. The system of claim 14, wherein the processor is communicatively coupled to the x-ray source and the detector, and wherein the x-ray source is automatically adjusted to the position indicated by the calculated angle.
